(19) United States
(12) Patent Application Publication
(10) Pub. No.: US 2024/0009476 A1
(43) Pub. Date: Jan. 11, 2024

(54) Transcranial Magnetic Stimulation (TMS) Apparatus

(71) Applicant: The Magstim Company Limited, Whitland Dyfed (GB)

(72) Inventors: Lothar Krinke, Whitland Dyfed (GB); Eric Granstrom, Whitland Dyfed (GB)

(73) Assignee: The Magstim Company Limited, Whitland Dyfed (GB)

(21) Appl. No.: 18/037,499

(22) PCT Filed: Dec. 31, 2021
(86) PCT No.: PCT/GB2021/053451

§ 371 Date: May 17, 2023

(30) Foreign Application Priority Data
Dec. 31, 2020 (GB) 2020811.2

(51) Int. Cl. A61N 2/00; A61N 2/02; G16H 40/67; G16H 10/20

(52) U.S. Cl. CPC A61N 2/006; A61N 2/02; G16H 40/67; G16H 10/20

(57) ABSTRACT

The present invention relates to a Transcranial Magnetic Stimulation (TMS) system comprising a TMS apparatus for delivery of TMS to a subject and an input device physically disconnected from the TMS apparatus. The TMS system is designed to enable use in a home environment without the requirement of an in person medical practitioner.

The present invention relates to Transcranial Magnetic Stimulation (TMS).

Magnetic stimulation of neuromuscular tissue is well known and comprises a stimulating coil made up of one or more windings, each having a plurality of turns which may generate a succession of electrical discharge pulses producing magnetic pulses which further induce electrical signals in the tissue. Such a coil arrangement is disclosed in U.S. Pat. No. 6,179,770 where the magnetic stimulator generally comprises a charging circuit, a capacitor, a discharge control and a winding which is of a size and power rating appropriate for the generation of magnetic fields sufficient to cause stimulation of a body portion. The individual winding or plurality of windings may be size adapted to fit partly over the cranium of a human patient in many applications, as well as being used for stimulation of other body parts. The coil arrangement including windings acts as an inductor and when connected to a stimulator which includes the capacitor provides an input voltage to the inductor which creates a circuit that passes an out of phase, sinusoidal voltage and current through the TMS winding. An intense sinusoidal magnetic field is formed near the winding and is used to stimulate neurons in patients for medical and research applications. A typical coil arrangement comprises a single winding coil arrangement that may suitably be positioned on a patient's cranium. The winding is made up of a single wound conductive element connected to the capacitor via an elongate neck that allows positioning of the coil arrangement appropriate to the patient.

Such TMS coil arrangements are used for both research applications and also medical applications, particularly in the treatment of depression. Accordingly, TMS treatment is performed in a clinical environment by trained medical professionals.

A challenge in the treatment of many mental conditions is a perception of a stigma associated with mental health disorders and/or with mental health care and treatment. Patients suffering from depression may feel direct or inferred social pressure that such conditions, care or treatment are undesirable; accordingly they may avoid diagnosis or treatment. In other cases, they may prefer the greatest degree of anonymity or privacy in their treatment.

Aspects of the present invention have been conceived with usability and the desire to utilise TMS treatment without the necessity to be in the presence of a medical practitioner.

According to a first aspect of the present invention there is a Transcranial Magnetic Stimulation (TMS system comprising a TMS apparatus for delivery of TMS to a subject and an input device physically disconnected from the TMS apparatus, the TMS apparatus comprising:
a coil comprising one or more coil windings wound from an elongate conductive element;
a stimulator for supplying electrical current to the one or more coil windings;
a control system for controlling the stimulator in delivery of a TMS treatment protocol to a subject, the control system comprising a communication unit configured to transmit information related to the subject and/or one or more operational parameters of the treatment protocol of the stimulator to the input device;

wherein the control system is further arranged to modify the treatment protocol in response to a control signal received from the input device.

A system is therefore provided that enables a subject to receive TMS treatment at a location remote from a medical practitioner or therapist, where the medical practitioner has access to an input device that is in communication with the control system. The treatment protocol for the subject/patient under the control of the control system of the TMS apparatus can be modified by a control signal from the remote device, meaning the treatment protocol can be operated with both safety and efficacy. The control signal is preferably dependent upon an input to the input device by a user (medical practitioner) remote from the TMS apparatus. In the context of the invention ‘remote’ means that the input device and TMS apparatus are physically separate, and will typically be located in at least different buildings.

It will be appreciated that the control signal is beneficially received into the communication unit of the control system.

A further advantage of the communication between the patient system and therapist system is that greater flexibility is afforded as a patient can flexibly schedule their therapy for “on-demand” treatment. For example, rather than having to fix an appointment for instance a week away, a patient may sit down at their treatment station, see the virtual presence of an attending therapist is confirmed as monitoring their end of a system, and start alignment/therapy “immediately” knowing that the attending therapist can readily monitor this new patient's therapy from afar “on demand.”

It will be appreciated that a remote location means there is no physical connection required between the input device and the TMS apparatus. Accordingly, the communication unit is preferably arranged to transmit the information (or signals representative thereof) wirelessly over the internet.

The stimulator beneficially supplies electrical current to the one or more coil windings in pulses, and wherein the operational parameters of the treatment protocol may comprise one or more of pulse duration, pulse repetition rate, total number of pulses in a treatment protocol and maximum electrical current supplied to the one or more coil windings during each pulse. A TMS protocol may be tailored for a particular subject and may involve optimisation for example of the number of pulses delivered, pulse intensity (current), pulse frequency for that particular subject.

A further benefit of the first aspect of the claimed invention is that such a TMS system enables a single therapist to, in parallel, support multiple TMS apparatus and thus subject treatments simultaneously; minimizing both required staff and required equipment for such therapy. The system may therefore comprise a plurality of TMS apparatuses in communication with a single input device physically disconnected from each TMS apparatus.

The communication unit is preferably arranged to transmit information related to the subject and/or one or more operational parameters of the treatment protocol of the stimulator to the input device during delivery of the treatment protocol. Even more preferably the control system is arranged to modify the treatment protocol in response to a control signal during delivery of the treatment protocol. Accordingly, a medical practitioner can monitor the information being transmitted relating to the subject undergoing treatment and make changes to the treatment protocol dependent upon their response to the treatment. As examples only, this could involve a subject stating either in message or verbal form that the treatment was causing discomfort and the medical practitioner could modify one or more treatment parameters. Alternatively, the treatment could be terminated. This allows a TMS apparatus to be used in a home environment in a safe and effective manner.

The control system may be arranged to modify the treatment protocol by either terminating and/or modifying supply of electrical current to the one or more coil windings by the stimulator. This can be completed responsive to a medical practitioner deciding to take the appropriate action. Whilst the TMS apparatus itself may have safety features to determine for example if a subject's head has moved a predetermined distance from a treatment position and thus terminate the treatment protocol, it is further beneficial that a medical practitioner also receives information relating to the subject and/or operational parameters of the treatment protocol. Appropriate action can therefore be taken to ensure safe continued treatment or treatment is terminated or paused.

Supply of electrical current from the stimulator to the one or more coil windings is preferably only enabled when there is operative communication between the communication unit and the input device. This provides a benefit in that the TMS apparatus cannot be operated by an unauthorised subject without the appropriate authorisation and monitoring by a medical practitioner. Accordingly, it is beneficial that for safety purposes the TMS system is arranged to maintain an operational connection between the communication unit and the input device, and only enable delivery of TMS to the subject when an operational connection is maintained.

The communication unit is preferably arranged to transmit the information during delivery of a treatment protocol to a subject, the treatment protocol comprising delivery of one or more pulses of electrical current from the stimulator to the one or more coil windings, and one or more parameters of the treatment protocol is controlled dependent upon the information received by the device. It is preferable that information is transmitted substantially continuously while a treatment protocol is underway, meaning that there is no significant delay in response to an unwanted event occurring such as the subject experiencing kindling.

The TMS system beneficially comprises a subject operable switch moveable between an on and off position for enabling and disabling supply of electrical current to the one or more coil windings, wherein the switch is arranged such that it must be actively maintained in an on position for the supply of electrical current to the one or more coil windings. This provides a further safety feature in that it means that the subject must effectively be holding the switch in the on configuration for treatment to occur. If the switch is released, then current will not flow and therefore treatment will not occur. It will be appreciated that the switch may take various forms. As an example, the switch may be in the form of what is referred to as a spring-loaded momentary switch operated for example by a thumb such that if the subject were to discontinue holding the button down (in the on-state) with their thumb (as might happen during a seizure, or if falling asleep, etc.) the switch would change position to an off-state. As a second example, the switch may take the form of a proximity/contact sensor where a subject must maintain hold a certain contact zone for the treatment protocol to be completed.

The TMS apparatus may comprise a camera for capturing visual images of the subject, and the information relating to the subject comprises captured image data. The captured images preferably comprise moving visual images. The input device preferably comprises a display screen for displaying captured images of the subject.

The provision of a camera (including audio functionality) provides numerous benefits. The information related to the subject transmitted to the input device may therefore be captured images of the subject. This allows significant functionality. There is beneficially two-way communication between the subject and the medical practitioner, meaning that conversation can take place and instructions provided to the subject. It is beneficial that the image data is transmitted during delivery of the treatment protocol as the medical practitioner can at this time modify the protocol (dependent upon the subject's visual/verbal response for example) or if necessary terminate the treatment protocol.

The camera and image data captured may also be utilised by the TMS apparatus itself without requirement for input from a medical practitioner. Accordingly, the image data may be used as an automated safety feature to give an indication that the subject experiencing ‘kindling’ which can result in a build up to a seizure. Based on this information the TMS apparatus may be arranged to terminate delivery of the treatment protocol immediately and further beneficially provide a notification to the medical practitioner via the input device. In order to achieve this operational benefit the control system is preferably configured to compare a captured image with a reference image of the subject, and if there is a predetermined difference between the captured image and the reference image the TMS treatment protocol is terminated. The control system may be arranged to identify one or more facial markers in the captured image, compare the one or more facial markers in the captured image to corresponding facial markers in the reference image, and if there is a predetermined difference between the positions of the one or more facial markers in the captured image to the facial image the TMS application treatment protocol is terminated. The facial markers may be determined with respect to a structure such as a receiving formation for receiving a subject's head (as defined with reference to the third aspect for example). Accordingly, the receiving formation can define a reference frame for the facial markers of the facial image, and if there is a predetermined difference between the position of the facial marker and the receiving formation then the treatment protocol can be terminated.

The input device preferably comprises a user operable input for receiving control instructions for the control system, and the control signal represents the control instructions. The input device may for example comprise a standard computing device with a screen able to communicate with the TMS apparatus.

Communication between the control system and the remote user input presents a security and safety risk for the subject with an associated risk of infiltration or disruption by outside unauthorized parties. Though inconvenient, disruption of TMS is unlikely to cause medical adverse events, making the use of a safety design requiring constant communication between the TMS apparatus and remote input device feasible. However, it is beneficial that the information (data) transmitted is protected. Accordingly, the communication unit is preferably arranged to encrypt the information before transmission to the input device.

As the input device and TMS apparatus are used in locations remote from one another, the communication unit may comprise a communication unit which is arranged to communicate with the input device over the internet. The communication unit is preferably arranged to communicate with the input device wirelessly. The communication unit may be arranged to communicate over a cellular network.

The TMS apparatus may further comprise a subject operable input device for inputting information relating to the subject. The subject can therefore communicate with a medical practitioner operating the input device to provide information relating for example operational parameters, and/or may comprise information relating to a subject perceived effectiveness of treatment or mood. The subject and medical practitioner preferably communicate via audio and visual data.

The TMS apparatus may comprise a monitoring arrangement for monitoring the subject during delivery of TMS to the subject and obtaining the information relating to the subject from the monitoring arrangement. The monitoring arrangement comprises an electroencephalogram (EEG) and/or electromyography (EMG) monitoring arrangement and the information related to the subject comprises EEG data and/or EMG data. EEG sensors may be positioned at one or more locations around a subject's head where the monitoring arrangement monitors and identifies output variations that exceed a predetermined threshold. Alternatively or in addition, an EMG apparatus may be utilised for monitoring one or more muscles and again the monitoring arrangement may monitor and identify output variations that exceed a predetermined threshold.

The apparatus is preferably configured to be operable only in the event of receipt of an acceptance signal from the input device at a remote location. This ensures that the TMS apparatus cannot be operated unless actively enabled by an authorised person at a remote location.

The TMS apparatus preferably further comprises a receiving formation for receiving a subject's head and for providing a reference frame for positioning the coil relative to a subject and a support structure for supporting the coil and receiving formation at an elevated position. The receiving formation primarily acts to ensure that the TMS coil is always positioned at an appropriate location on the head for each treatment, but can also be utilised for safety purposes in providing a reference frame based on which the relative position of facial features can be determined and operation of the TMS apparatus can be modified depending on the relative position of those facial features. For example if the subject has moved too far from the required treatment position then the TMS treatment protocol is either termination or not allowed to start.

The receiving formation preferably comprises a first contact portion for contacting a first area of a subject's head, a second contact portion for contacting a second area of a subject's head, and an intermediate portion extending between the first and second contact portions. The support structure further comprises the stimulator.

Also according to a first aspect of the present invention there is a method of controlling delivery of Transcranial Magnetic Stimulation (TMS to a subject comprising providing a TMS system according to any preceding claim and comprising the steps of:
transmitting information related to the subject and/or one or more operational parameters of the treatment protocol of the stimulator to the remote input device;
inputting TMS control instructions to the remote input device dependent on the transmitted information and transmitting a control signal to the communication unit of the TMS apparatus;
modifying the treatment protocol in response to the control signal received from the input device.

The method preferably further comprises the step of a subject inputting information relating to the subject.

The method preferably further comprises the step of establishing an operational connection between the TMS apparatus and the input device, and only enables delivery of the treatment protocol when the operative connection is maintained.

The information related to the subject and/or one or more operational parameters of the treatment protocol of the stimulator and the control signal are beneficially transmitted wirelessly over the internet.

The transmitted information may comprise captured image data.

In most TMS systems, access to system controls is afforded through entry of authentication information by one person—an attending medical practitioner/therapist. In a preferred embodiment, authentication is entered by two parties—both the attending therapist via the input device and the subject undergoing the treatment via the TMS apparatus.

According to a second aspect of the present invention there is a Transcranial Magnetic Stimulation (TMS system comprising a TMS apparatus for delivery of TMS to a subject and an input device physically disconnected from the TMS apparatus, the TMS apparatus comprising:
a coil comprising one or more coil windings wound from an elongate conductive element;
a stimulator for supplying electrical current to the one or more coil windings;
a control system for controlling the stimulator in delivery of a TMS treatment protocol to a subject;

wherein the system is arranged to establish and maintain an operational connection between the communication unit and the input device, and only enable delivery of TMS treatment to the subject when an operational connection is maintained.

This provides a benefit in that the TMS apparatus cannot be operated by an unauthorised subject without the appropriate authorisation and monitoring by a medical practitioner. Accordingly, it is beneficial that for safety purposes the TMS system is arranged to maintain an operational connection between the communication unit and the input device, and only enable delivery of TMS to the subject when an operational connection is maintained.

An operational connection is beneficially established in response to the control system receiving an authentication signal from the remote input device. This authentication signal is preferably in response to a request signal transmitted from the control system (following input from a subject) of the TMS apparatus to the remote input device.

The TMS system preferably further comprising a camera for capturing moving visual image data of the subject and transmitting the moving visual image data to the TMS apparatus.

The control system is preferably arranged to modify the treatment protocol in response to a control signal received from the input device.

The TMS apparatus preferably comprises a subject operable switch moveable between an on and off position, wherein the switch is arranged such that it must be actively maintained in an on position for an operational connection to be maintained.

The second aspect of also extends to a method of controlling operation of a Transcranial Magnetic Stimulation (TMS), the method comprising providing a system according to the second aspect and establishing and maintaining an operational connection between the communication unit and the input device, and enabling delivery of TMS treatment to a subject only when an operational connection is maintained.

It will be appreciated that features and functionality together with the advantages provided described with reference to the first aspect of the invention are equally applicable to the second aspect. With respect to both aspects it will be appreciated that the input device is in a location remote from the TMS apparatus. This means that the TMS apparatus can be located in a home environment and the medical practitioner at another location such as a clinic with the ability to communicate with multiple individuals.

There are further problems associated with TMS use, both in research and medical environments. A further problem exists of positioning the coil arrangement appropriately. There are many locations that are used to treat various medical conditions that involve TMS for example over the occipital nerve for the treatment of migraines and the dorsolateral prefrontal cortex (also known in the field as F3) for the treatment of major depressive disorder. Finding a particular treatment location is difficult and the efficacy of treatment is highly dependent upon locating and treating the intended area. As an example, F3 can be achieved by using the so-called ‘Beam F3 method’ or the ‘5.5 cm’ method. In this example the 5.5 cm method is described.

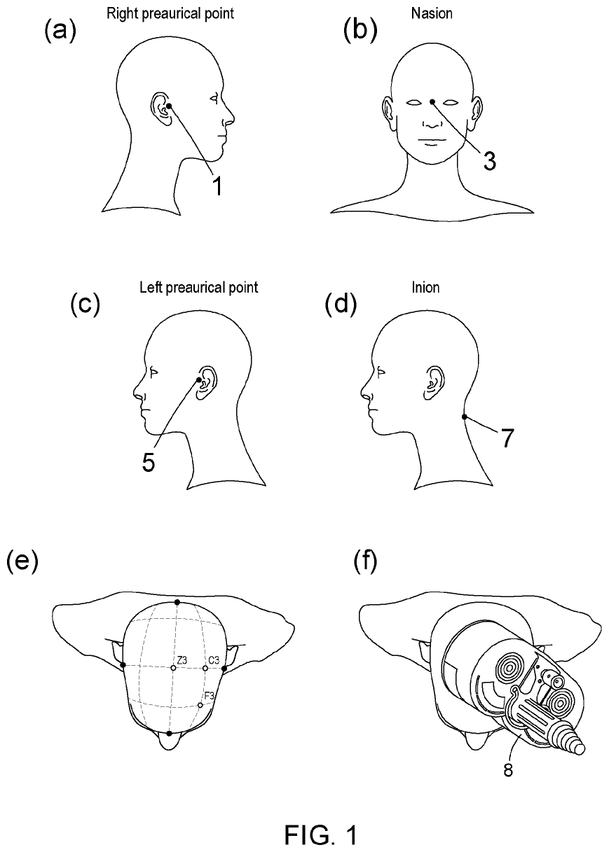

The intention is to initially position the TMS coil over the motor cortex area of the brain associated with the right thumb (C3), from which the F3 location can be found. FIG. 1 shows the four bony landmarks on the head in FIG. 1a-d used to determine important landmarks to generate a head model based on these reference points. These are the right preauricular point 1, nasion 3, left preauricular point 5 and inion 7 respectively. FIG. 1(e) shows a position known as 73′ which is found by marking the mid distance from the nasion to inion adjusted by the mid distance from the right to left preauricular points. C3 is found by marking a point 20% of the distance from the right to left preauricular points. A procedure involving power adjustment and fine movements of the coil is followed until a thumb twitch is observed when the coil is pulsed over the patient specific area for the thumb found from the motor mapping starting from C3. This represents the minimum power setting to stimulate the cortex. This point is then typically manually marked by some means and then the treatment location is determined by moving the coil 5.5 cm forward from C3 to F3. Once this location is appropriately identified, an indicator is placed on the patient's cranium at F3 and the coil 10 directed to this location as presented in FIG. 1(f).

However, this is difficult to achieve as the coil arrangement obscures the view of the operator. TMS operators take a great deal of time and effort to ensure that the TMS coil is correctly placed in the correct location and to do this as in the manual method described above is costly in time and therefore there is a need for a better system. In addition, getting the coil quickly onto the same treatment spot accurately on subsequent patient visits is the key to an efficient clinical practice.

Navigation systems have therefore been developed to achieve easier coil arrangement positioning. A relatively simple method is to provide a patient with a cap which is worn on the head. At the first treatment the appropriate position for treatment is located and marked on the cap in the known way of finding the motor threshold and treating at a predetermined location from this. On subsequent treatment sessions the cap is worn, and treatment carried out at the same location as marked. However, such a technique is susceptible to the cap being incorrectly positioned on the head. In addition, as the TMS coil is large the operator cannot see the underside of the coil and it is difficult to ensure that the point between the two windings in the TMS coil arrangement is actually positioned in alignment with the treatment location. Accordingly, treatment may be carried out at the wrong location thereby reducing efficacy.

Other techniques include infrared tracking cameras that track reflective markers positioned on the coil arrangement and on the patient's head. However, in use reflective markers may lose their reflective coating potentially causing error in measurements. Furthermore, interference is common through changes in light conditions such as sunlight entering a room, and in addition the required cameras are high cost.

A simple, low cost and reliable system is therefore needed for appropriately positioning a magnetic stimulation coil arrangement, and preferably a TMS coil arrangement, preferably into a treatment position.

According to a third aspect of the present invention there is Transcranial Magnetic Stimulation (TMS) apparatus comprising:
a coil comprising one or more coil windings wound from an elongate conductive element;
a receiving formation for receiving a subject's head and for providing a reference frame for positioning the coil relative to a subject; and
a support structure for supporting the coil and receiving formation at an elevated position.

Irrespective of the location or orientation of the support, the reference frame is always the same with respect to the receiving formation and accordingly the first and second areas of a subject's head. This means that the coil can be repeatedly positioned in the reference frame, and the appropriate location on the subject's head can be treated.

The receiving formation preferably comprises a first contact portion for contacting a first area of a subject's head, a second contact portion for contacting a second area of a subject's head, and an intermediate portion extending between the first and second contact portions. The provision of the first and second contact portions and intermediate portion extending therebetween improves the accuracy and consistency of the reference frame. The reference frame is therefore preferably always the same with respect to the first and second areas of a subject's head.

The receiving formation is preferably arranged to receive a subject's head such that the subject leans forwardly into the reference frame. The receiving formation preferably defines a receiving zone open in a generally downward orientation when in an operative configuration. This means that rather than in a traditional TMS apparatus where a subject tilts their head rearwardly to be received into a headrest, the subject lowers their head into the receiving zone. This reduces the chance of a subject accidentally moving out of the treatment position. When in this position, the subject provides support to themselves on a support surface on which the TMS apparatus is sited. Typically, the subject will support themselves on their elbows.

The position of the coil relative to the receiving formation is beneficially predetermined by a technique as described for example above.

Such an apparatus is particularly beneficial for a TMS apparatus that can be used without the presence of a medical practitioner. As the reference frame is consistent with respect to an individual subject's head, the appropriate location for the coil can be readily determined.

The receiving formation may comprise a third contact portion for contacting a third area of a subject's head.

The first and second areas are distinct areas of the subject's head. For example, the distinct areas may be a subject's chin and ear. The first and second contact portion may each comprise respective first and second contact surfaces, wherein beneficially at least a portion of each of the first and second contact surfaces are oriented at an angle of at least degrees relative to each other. Through the relative angle between the first and second contact surfaces a good fit to the subject is achieved where the equal and opposite force acting on the subject means that the reference frame can be more consistently achieved compared to more linear contact surfaces. It is even more beneficial that at least a portion of the first and second contact surfaces are substantially orthogonal to one another.

The first contact portion is preferably arranged to contact a subject's chin or nose. The first contact area preferably receives a subject's chin, nose or forehead. One embodiment of such a system may be one in which a chin support is constructed to bear a sizable weight load from the patient. The second contact portion is preferably arranged to contact a subject's ear. By providing contact with a subject's chin/nose and ear, the accuracy of positioning of the coil can be increased. The particular combination of chin/nose and ear positioning results in a subject's head being able to be placed into an accurate reference frame which is specific to a particular subject. Accordingly, for each treatment, a subject will be able to ensure that a treatment area for the coil can be readily located.

The second contact portion may be shaped to at least partially seat into a subject's ear. Thus, the second contact area may comprise a subject's ear. This is beneficial as increases the accuracy of the location of the receiving formation and thus the reference frame for accurate location of the coil. The second portion may further comprise a speaker for outputting sound to the subject. For example, the sound may be operational instructions or information relating to use of the apparatus. The second contact portion may muffle the sound produced by the coil in operation resulting in a more pleasant experience for the subject.

The first and/or second contact portions may be customised to fit to a specific subject. For example, the first and/or second contact portions may be moulded to follow the contours of a subject's head, and optionally be formed to receive a particular subject's chin and/or ear. The first and/or second contact portions may be demountable relative to the intermediate portion. This enables ease of customised first and/or second portions to be mounted to the apparatus as required.

It will be appreciated that the intermediate portion is rigid, meaning there is substantially no deflection of the intermediate portion, thus ensuring consistency of the relative position between first and second contact portions.

The support structure preferably further comprises at least one articulation arrangement for enabling raising and lowering of both the coil and receiving formation between the elevated and a lowered position. This enables ease of use for a subject independently as a subject can manipulate the apparatus to a position comfortable for their personal use.

The support structure preferably further comprises a base for seating onto a raised surface. The base may comprise or include a stimulator for supplying and controlling current flow to the coil.

The receiving formation may be designed to allow the patient to rest nominally “on their elbows” during treatment to reduce pressure on the chin, forehead, head, etc., which is of particular importance with a treatment not occurring on an otherwise highly engineered medical patient therapy chair such as in a home environment. Likewise, the receiving formation may be designed to allow the patient to nominally rest a good portion of their body mass into or onto the system (i.e. incline forward into a system) rather than a traditional design in which the patient is reclined.

The support structure preferably comprises an adjustment mechanism for adjusting and fixing the relative position between the coil and the receiving formation. A guide for indicating to a subject the relative position between the coil and receiving formation may be provided. This can be, for example, visible adjustment markers or may alternatively comprise information presented by visual or sound to the subject. This is important as the guide ensures that the coil is correctly positioned with respect to the required and typically precise treatment location.

The coil may be moveable relative to the receiving formation. The coil may be moveable between an open configuration and an operational configuration. In the open configuration a subject is able to position their head to be received by the receiving formation. In the operational configuration, the coil is in a position to apply treatment to a predetermined location on the subject. The position of the coil in the operational configuration is beneficially predetermined, meaning that a subject does not have to spend time aligning or selecting the treatment position—it is beneficial that movement of the coil is restricted to movement to a predetermined operational configuration.

The support structure preferably further comprises a display arrangement for displaying information relating to operation of the TMS apparatus. The display arrangement is preferably integrated into the support structure. Preferably the apparatus further comprises an input device for inputting information relating to operation of the device. The information input may for example be information relating to the user and/or operational parameters. The display arrangement and input device may be combined into a single touch screen for example.

The support structure preferably further comprises a stimulator for supplying current to the one or more windings.

Known TMS systems typically comprise a chair having some sort of receiving formation such as a headrest that is adapted hold a user's head reasonably still. A stimulator is carried on a moveable trolley or similar and extending from this stimulator is a support arrangement that can carry the coil. The coil is usually suspended from the support arrangement. When treatment is to be performed, the coil is brought to the appropriate position relative to the subject and locked in position. Such an arrangement occupies significant space and is complex to use, requiring skilled operators.

Accordingly, the support structure preferably comprises a housing having a stimulator therein for supplying electric current to the one or more coil windings and a support arm arrangement extending therefrom for supporting the coil and the receiving formation.

The TMS apparatus is beneficially a desktop apparatus. This is particularly beneficial in that instead of a subject having to attend a clinic for treatment, the apparatus can be used outside of a clinic such as at home. As the coil and receiving formation are supported by the stimulator housing, the size of the apparatus can be significantly reduced and usability increased.

Further, such a TMS apparatus is beneficially a portable system, designed for frequent setup and breakdown, and preferably with a full system weight of less than 20 kg.

The support arm arrangement, which may comprise one or more support arms, preferably extends intermediate the housing and both the coil and receiving formations.

The housing or stimulator housing preferably comprises a base. The base may be configured to sit onto a raised platform such as a desktop.

The support arm arrangement may take various forms. The support arm arrangement is preferably arranged to enable the height of the coil and receiving formations to be adjusted dependent on the height required for a patient. The support arm arrangement may comprise multiple linkages thereby enabling movement of the coil and receiving formation relative to the housing. The support arm arrangement may comprise one or more support arms. The support arm arrangement is preferably configurable such that the receiving formation and coil are moveable between an operational configuration and a stowed configuration. In the stowed configuration the support arm arrangement and accordingly the receiving formation and coil are preferably adjacent to the housing.

The support arrangement preferably further comprises a display arrangement for displaying information relating to operation of the TMS device. Preferably, the apparatus further comprises an input device for inputting information relating to operation of the device. The information input may be for example information relating to the user and/or operational parameters. The display arrangement and input device may be combined into a single touch screen for example. The display arrangement is preferably integrated into the stimulator housing.

Also according to the third aspect of the invention there is use of a TMS apparatus according as hereinbefore described wherein the support structure is supported on an elevated support surface. A subject leans forwardly into the receiving formation and at least partially supports the weight of their upper body on the elevated support surface with their forearms.

Also according to the present invention there is a method of positioning a Transcranial Magnetic Stimulation (TMS) apparatus relative to a subject comprising:
providing a reference frame for positioning a coil relative to the subject by positioning a first area of a subject's head in contact with a first contact portion of a receiving formation and positioning a second contact area of a subject's head in contact with a second contact portion of the receiving formation; and
positioning the coil at a predetermined treatment location in the reference frame.

The method preferably comprises a setup step whereby the predetermined treatment location is found or selected. This can be achieved by using known techniques, such as by the 5.5 cm method described above. This predetermined treatment location is specifically identified for a particular subject. It is beneficial that an intermediate portion extends between the first and second contact portions.

According to a further aspect of the present invention there is a positioning arrangement for enabling positioning of a TMS coil relative to a patient's head, the positioning arrangement comprising first and second contact portions for receiving a subject's head at a first and second contact area respectively and providing a reference frame for positioning a coil relative to the subject, the positioning arrangement further comprising a support structure for supporting the first and second contact portions, wherein at least the first contact portion is mountable to the support structure.

The first and second contact portions are preferably both releasably mountable to the support structure. The first and/or second contact portions are preferably also demountable from the support structure.

The first and second contact areas are distinct areas as described above.

The first and/or second contact portions are therefore customised to fit to a specific subject. For example, the first and/or second portions may be moulded to follow the contours of a subject's head, and optionally be formed to receive a particular subject's chin and/or ear. A significant benefit is that when used repeatedly a subject is always held in the same way with respect to first and second contact positions. This means the reference frame relative to these two points is consistent, and the coil can more accurately be positioned into a predetermined treatment location.

The first and preferably second contact portions may comprise first and second contact pads.

The first and second contact portions may be constructed of a closed or open cell structure. A suitable contact pad may be constructed for example of a foam material.

The first and preferably second contact portions are secured to the support arrangement by a securing arrangement. Such a securing arrangement may comprise for example cooperating clips or similar. There are multiple suitable options for securing of the contact portion(s) to the support arrangement.

The first and second contact portions may be independent of one another or integrally formed.

Also according to this aspect of the invention there is a method of positioning a TMS coil relative to a subject's head using a positioning arrangement, where the positioning arrangement comprises first and second contact portions for receiving a subject's head at a first and second contact area respectively and provides a reference frame for positioning a coil relative to the subject, wherein the method comprises recording the contours of at least the first contact area of the subject and using the recorded contours to form a customised first contact portion, and mounting the customised first contact portion to a support structure for supporting the first and second contact portions.

A significant benefit of aspects of the present invention are that treatment may occur with the subject/patient in complete physical isolation, a means of affording both anonymity and privacy, as well as a great benefit for patients in various physically constrained circumstances, including but not limited to perceived immunological risk, such as during COVID-19 related travel and distancing restrictions. Furthermore, a TMS system may be used in which communication with the patient during treatment is entirely indirect, meaning no traditional direct communication (speaking, gesturing, etc.), but is entirely electronic (e.g. video or audio broadcasts).

Illustrative embodiments of the present invention will now be described by way of example only with reference to the accompanying Figures where:

FIG. 1a-f are representations of reference points used for determination of a treatment position represented in FIG. 1e, and FIG. 1f includes a TMS coil;

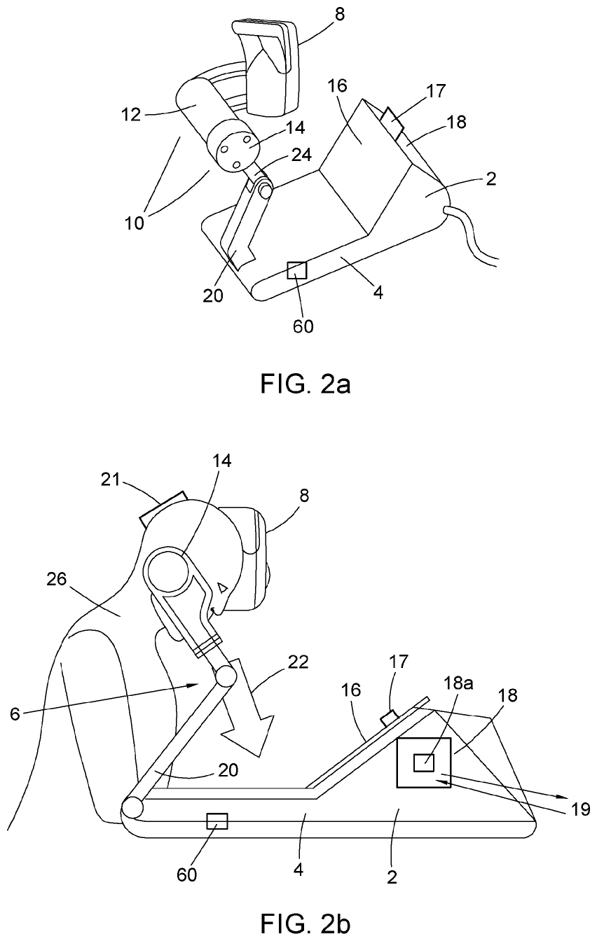

FIG. 2a is a schematic perspective representation of a TMS apparatus according to an illustrative embodiment of the present invention, and FIG. 2b is a schematic representation of the same apparatus with a subject in a treatment position;

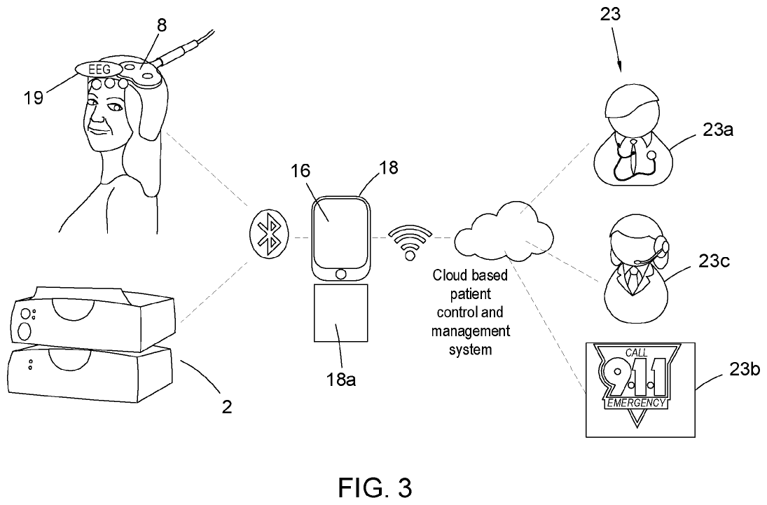

FIG. 3 is a schematic representation of an overall system architecture according to an illustrative embodiment of the present invention;

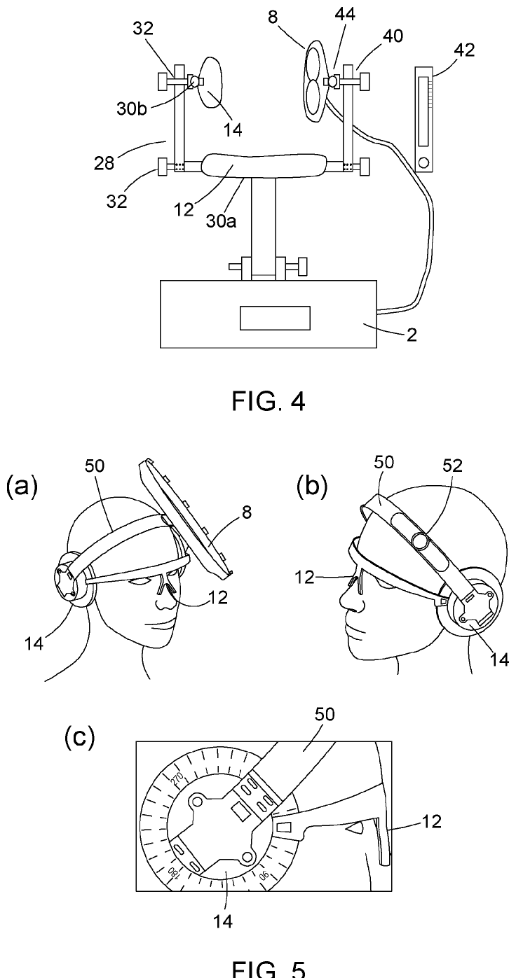

FIG. 4 is a schematic perspective representation of a further TMS apparatus according to an illustrative embodiment of the present invention;

FIG. 5a-c are schematic perspective representations of a further TMS apparatus according to an illustrative embodiment of the present invention; and

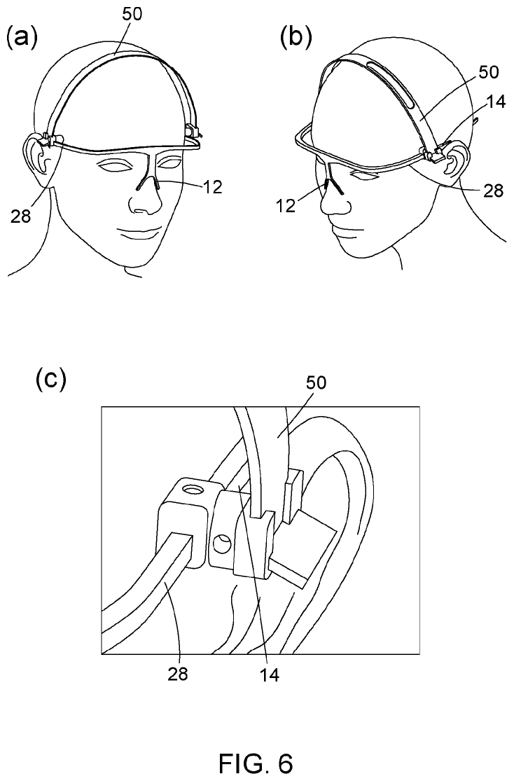

FIGS. 6a-c are schematic perspective representations of a further TMS apparatus according to an illustrative embodiment of the present invention.

Referring to FIGS. 2a and 2b, there is a schematic representation of a TMS apparatus according to an illustrative embodiment of the present invention. The apparatus comprises a stimulator 2 provided within a housing 4 designed to sit onto an elevated surface such as a desktop. Extending from the housing 4 is a support arm arrangement generally identified by reference numeral 6 projecting upwardly such that the apparatus is in an operational configuration. The support arm arrangement 6 provides support to both the coil 8 and the receiving formation 10 made up of a first contact portion 12 and second contact portion 14. The apparatus is compact and user-friendly meaning that it can be operated by the subject undergoing the treatment.

In more detail, the stimulator 2 provided within the housing 4 forms a base for the apparatus and includes a control system 18 that controls delivery of a treatment protocol to a subject. The apparatus is portable meaning that the apparatus can be brought out for use and stored away after use. Beneficially provided within the housing 4 is a display arrangement 16 forming part of the control system 18 having a screen which is able to provide both information to a subject undergoing treatment and also be capable of receiving inputs from a subject to communicate with a remote input device 19. For example, the display arrangement 16 (which may also accept user inputs) may provide indications to a subject of operational parameters such as intensity of treatment or time remaining or may provide prompts to a user to provide an input at regular intervals to ensure the subject is comfortable and is not feeling any unwanted side effects. This may be automated as described below, where the treatment is automatically stopped in the event that a potential seizure is detected under control of the control system 18. The control system 18 further comprises a communication unit 18a capable of two-way communication with the remote input device 19. The TMS apparatus further comprises a camera 17 for capturing image data of the subject particularly but not exclusively when the subject is undergoing treatment.

The remote input device 19 can be located anywhere provided there is communication with the control system 18. Accordingly, the remote input device can communicate with multiple individual TMS apparatuses. The remote input device 19 is capable of communication with the communication unit 18a of the TMS apparatus and comprises a computing device. It is capable of communication with the TMS apparatus and once a connection is established and maintained then the TMS apparatus can be operated. This can be achieved for example by a subject providing an authentication input and a medical practitioner providing an authentication input thereby allowing continued communication between the TMS apparatus and the remote input device. In order to ensure that a subject remains in position for treatment, there may be provided a switch 60 such as a contact sensor, proximity sensor, or other suitable switch that must be both activated by the subject and physically maintained in the activated configuration for the TMS treatment protocol to be delivered. The switch 60 may be positioned such that it is comfortable for contacting the patient during operation. For example the switch 60 may be positioned in one or both of the first and second contact portions 12,14. The switch functionality is described in more detail below.

The TMS system as described can be configured and operated in various ways to depending on the degree of input and control required from a remote location. The following functionality will be described separately however it will be appreciated that various functions can be provided in combination with other functions. The system is designed however to enable use in the home without the physical presence of a medical practitioner. For this reason, monitoring of the subject during treatment is important.

Once a connection between the TMS apparatus and the remote input device 19 is established and maintained, a treatment protocol can be delivered. The control system may be arranged to modify the treatment protocol in response to a control signal received from the remote input device 19 which may be determined based on information relating to the subject or operational parameters. In one embodiment, prior to delivery of the treatment protocol the remote input device 19 receives data comprising information related to the operational parameters of the TMS apparatus. The medical practitioner can then determine the appropriateness of the treatment protocol and then make changes as deemed necessary perhaps based on previous treatments or the patient's response history. This modification may occur before, during, or after treatment protocol delivery.

Alternatively or in addition, once a connection is established, information relating to the subject in addition or separate to the operational parameters is transmitted to the remote input device which is monitored by a medical practitioner and modification to the treatment protocol may be made. This information may take various forms. It is particularly important in TMS treatment that a subject is monitored in case of undergoing a seizure. This is a potentially serious issue, especially when the present invention is designed to be capable of being used in a subject's own home without medical supervision. In certain circumstances this may be because of the effect of ‘kindling’, which is the brain responding to synchronisation with the TMS treatment which can result in a build up to a seizure. A camera for receipt of moving visual image data of the subject is beneficial, and this image data is displayed on a display screen of the remote input device 19. Accordingly, a medical practitioner can monitor the subject during treatment and modify the treatment protocol during delivery. Modification may mean a change to the treatment protocol, or to stop the treatment protocol as required. In the event stopping of the treatment protocol is required, then the medical practitioner may need to alert a local practitioner or the emergency services if for example the subject could or is showing signs of kindling. The provision of such a camera allowing video call capability is beneficial as it also provides reassurance to the subject undergoing the treatment that the medical practitioner is monitoring the treatment protocol in a similar manner as they would if they were in the same room as the subject.

Alternatively, or preferably in addition as shown in FIG. 2b a monitoring arrangement 21 may also be utilised to actively monitor the subject during treatment and output information to the remote device 19 during delivery of the treatment protocol. Single or multiple different monitoring arrangements may be utilised. An illustrative monitoring arrangement 21 may comprise EEG sensors to be positioned at one or more locations around a subject's head where the monitoring arrangement monitors and identifies output variations that exceed a predetermined threshold. Alternatively, or in addition, EMG apparatus may be utilised for monitoring one or more muscles and again the monitoring arrangement monitors and identifies output variations that exceed a predetermined threshold. A control signal is then transmitted from the remote input device 19 to modify the treatment protocol.

The control system 18 is operable to alter the treatment protocol dependent upon the information transmitted to the input device 19. The treatment protocol may comprise one or more parameters to include frequency between pulses of energy, pulse duration, total number of pulses, pulse intensity, pulse train duration and/or number of pulses in a pulse train. Any one or more of these parameters may be changed depending on information received by the input device 19. Altering of the treatment protocol may take various forms. Altering the treatment protocol may comprise terminating the treatment responsive to predetermined output information from the monitoring arrangement. For example, the output information may comprise an indication that the subject experiencing ‘kindling’ which can result in a build up to a seizure. In this circumstance it is important that treatment is terminated immediately.

Once the information has been transmitted and received by the remote input device 19, a medical practitioner can then analyse the information received. From this, a medical practitioner can then input information to the remote device and a signal representative of that information is returned to the communication module 21 of the TMS apparatus. Based on this returned information the control system 18 is configured to control operation of the TMS apparatus through modification of the treatment parameters for example. Alternatively or in addition, the medical practitioner can provide output information to the subject such as via onscreen or audible instructions. This provides a significant benefit as the subject can receive personalised instructions and can also receive a treatment protocol directly dependent upon a subject's response to treatment, either measured or based on a subject's own feeling.

The TMS apparatus preferably further comprises an input arrangement which may be the screen 16 for receipt of a subject input comprising information relating to the subject. The input may comprise information from the subject such identification information, a request for treatment to commence to be sent to the remote device, and/or may comprise information input by the subject relating to a subject perceived effectiveness of treatment or mood.

An additional feature of the present invention is that the TMS apparatus itself may also perform automated monitoring of the subject during treatment by the TMS apparatus itself. This may utilise the camera, where the control system is configured to determine a predetermined change in the appearance of a subject from the image data through for example identification of facial features and the relative position of the facial features relative to the receiving formation 10. The control system 17 comprising system software analyses the images output and from these images determines a predetermined change in the appearance or position of the facial features of a subject. Such a change may for example be a predetermined change in position of the subject indicative of a subject for example slumping in their seat, and if the position of a facial feature exceeds a predetermined distance then treatment can be automatically terminated.

The TMS control system 18 may be constructed such that confidential or sensitive patient information (including but not limited to personally identifiable information or treatment information) is only stored temporarily in the patient-local control system (such as would be the case if stored in traditional computer Random Access Memory (RAM)) such that by system design, such information is irretrievably lost from the local environment after immediate use, greatly reducing the risk that a patient-control system could intentionally or unintentionally care such confidential or sensitive patient information to an unauthorized party (such as, for example, a different future patient using the same instrument in their own home).

It is important that the TMS apparatus is used safely by the appropriate person where the risk of unsafe or improper use is increased in an environment where a medical practitioner is not necessarily present in the same room. Accordingly, there may be three safety features: use of an enabling switch/button 17 by a patient, use of an enabling button by a therapist, or use of an enabling button by both patient and clinic. An enabling button at the patient ensures safety against a variety of undesired situations—a patient might depart leaving the system available for others to use, a patient may suffer from an adverse medical event, or even more simply, a patient may be distracted and, in that distraction, shift position and/or be engaged in another activity that should not be disrupted. An enabling button at an attending therapist (clinician, technician, attendant, supervisor, etc.) affords similar safety. Though in a preferred environment the attending therapist should have visual confirmation of the patient's status throughout treatment (potentially via a camera arrangement), the same sorts of risks for attending therapist exist during treatment (departure, adverse event, distraction, etc.). The use of an enabling switch on both ends requires a conscious/continuous affirmation of intent to continue operation and affords a great degree of system safety.

The use of an enabling switch may be direct and literal. It may include a discrete physical button at the patient or at an attending therapist, such as in a handheld remote button. Alternatively, it may comprise a virtual button on a touch screen. It may also be entirely virtual in the sense of a presence or contact sensor at a patient confirming the patient hasn't left the instrument or had a head fall out of alignment, or similarly that an attending therapist hasn't left a console designed for supervising treatment. In these latter cases, the “enabling switch” may simply be a configured “feature” in hardware, firmware or software within the system.

In an embodiment and with reference to FIG. 3 there is a system the communication unit transmits information related to the subject and/or operation of the apparatus to a remote location. The communication module may comprise computing device 18. In the event of an indication that the subject may be experiencing kindling for example it is important that in addition to termination of the treatment, information is also transmitted wirelessly via a cloud based control and management system to a remote location 23 such as a medical practitioner 23a or in severe cases to alert emergency services 23b. In severe cases leading quickly to a seizure, in the event the subject is undergoing treatment alone, then emergency services 23b should be quickly notified. The control system may also be arranged to emit an alarm signal to a remote location in the event of determination of an adverse event. The alarm signal may also comprise an indication from the apparatus, for example an audible or visual alarm. The control system may further enable contact with technical support 23c as required.

Referring further to FIG. 2, the apparatus further comprises a support arm arrangement 6. In the embodiment presented in FIG. 2 the support arm arrangement 6 comprises a primary arm 20 pivotally mounted relative to the housing 4. Arm 20 is moveable indicated by arrow 22 in FIG. 2b to enable ease of storage and primary height adjustment relative to the surface onto which the apparatus is situated. A secondary arm 24 is pivotally mounted relative to the primary arm 20 further enabling height and incident angle upon the subject 26 as schematically presented in FIG. 2b to be adjusted. In the illustrative embodiments of FIGS. 2a, 2b and 4, the receiving formation 10 is made up of a first contact portion 12 for contacting a subject's chin and a second contact portion 14 for contacting a subject's ear. An intermediate portion 28 clearly shown in FIG. 4 extends between the first and second contact portions 12, 14.

Reference is now made to FIG. 4. The first and second contact portions 12, 14 can be positioned in fixed positions relative to one another. In this embodiment, the contact portions can be personalised for a particular subject by moulding or otherwise forming from a material such as foam or gel to accommodate the shape of an individual subject. This ensures that the subject is accurately and repeatably framed or located with respect to the first and second contact portions meaning that as long as the coil is repeatably positioned with respect to the first and second contact portions then the predetermined treatment location is always treated. The first and second contact portions may be each carried by a respective carrier 30a, 30b which can be releasably mounted to the remaining support structure. As presented in FIG. 4, an intermediate portion 28 extends between the first and second contact portions 12,14. Alternatively or in addition, the intermediate portion 28 may comprise one or more adjusters 32 as shown in FIG. 4 for enabling adjustment of the relative position of the first and second contact portions 12,14. The adjusters 32 allow for reconfiguration between a release configuration and a locked configuration, meaning that the positions of the first and second contact portions can be adjusted.

The second contact portion 14 preferably locates in communication with a subject's ear. This is beneficial as a subject's ear has limited movement relative to the rest of the head and therefore provides a good fixed location on a subject's head. It is preferable that the second contact portion 14 locates into a subject's ear, or receives a portion of a subject's ear therein, further increasing accuracy of positioning of the second contact portion. The second contact portion 14 may further comprise a speaker for transmitting audio to a subject. The audio may comprise information relating to the treatment such as instructions. Alternatively, the audio may be selectable according to a subject's preference such as music for example whilst the treatment is being performed. The second contact portion 14 may also reduce the sound being transmitted to the subject during operation. The second contact portion 14 may include acoustic insulation.

The first contact portion 12 utilises a fixed location on a subject and uses that location in the provision of a reference frame for positioning the coil 8. As presented in FIGS. 2b and 3 the first contact portion 12 contacts and preferably receives a subject's chin, whereas in the illustrative embodiment of FIG. 5 the first contact portion 12 contacts and preferably receives the bridge of a subject's nose. Both optional locations are selected for their consistency, and the fact that there cannot be significant variation in how a subject contacts or engages the contact portions with these facial features. Beneficially, the difference in orientation between the first and second contact locations ensures that a subject's head seats into the receiving formation in a fixed orientation. In the illustrative embodiments the two contact locations (e.g. chin and ear, or nose and ear) provide support in generally orthogonal axes meaning that a subject's head is comfortably and consistently received.

The first and second contact portions 12, 14 together define a reference frame for positioning of the coil 8 relative to a subject. Accordingly, providing a subject positions their head in a consistent position with respect to the first and second contact portions 12, 14 then the appropriate location can be treated.

The coil 8 can be moveable relative to the receiving formation 10 between an open configuration and an operational configuration. In the open configuration a subject is able to position their head to be received by the receiving formation. In the operational configuration, the coil 8 is in a position to apply treatment to a predetermined location on the subject. The coil 8 in the operational configuration may also act to restrict significant movement of the head to further ensure that treatment is being performed on the appropriate location. The position of the coil in the operational configuration is predetermined, meaning that a subject does not have to spend time aligning or selecting the treatment position. It is beneficial that movement of the coil is restricted to movement between a single open configuration, then is moved straight to a predetermined operational configuration. The operational position is predetermined by known techniques and preferably during a setup procedure by a medical practitioner.

In order to achieve movement of the coil 8 relative to the receiving formation 10 and therefore be adjustable to the appropriate location in the reference frame, the support structure comprises an adjustment mechanism 40 for adjusting and fixing the relative position between the coil 8 and the receiving formation 10. This may be achieved through various means. In one embodiment for example as presented in FIG. 4 adjustment to the appropriate location can be achieved using simple markers 42 (schematically presented in side view in FIG. 4) and ball and socket adjusters 44. Alternatively, movement of the coil 8 may be automated utilising a controller and motor where the coil 8 is moved to the predetermined location upon a user input to the display arrangement 16 for example.

In an embodiment, the location of the coil 8 can be set in a set up stage and can be prevented from subsequent movement by a subject who will undergo the treatment. In this way once the set up stage is performed, a subject will not subsequently adjust positioning of the coil 8 but will merely position themselves in the appropriate orientation with respect to the first and second contact portions 12, 14 meaning that the coil 8 is appropriately positioned.

In alternative embodiments, adjustment can be made to the positioning of the coil 8 after a subject positions themselves with respect to the receiving formation 10. In such an embodiment, movement limiters such as stops may be included to limit the range of movement and ensure that the coil 8 is moved to the appropriate predetermined treatment location. A guide for indicating to a subject the relative position between the coil and receiving formation is preferably provided. This can be for example visible adjustment markers 42 or may alternatively comprise information presented by visual or sound to the subject (e.g. via display arrangement 16 and/or the second contact portion 14). This is important as ensures that the coil is correctly positioned with respect to the required and typically precise treatment location.

In setup of the apparatus in preparation for application of TMS to a subject, unless the first and second contact portions 12,14 are shaped specifically for a particular subject, the relative positions of the first and second contact portions 12,14 are adjusted to fit to the subject. For example, the second contact portion 14 is positioned into a user's ear, whilst the first contact portion 12 is adjusted to receive the user's chin. These positions are then fixed and are specific to that particular subject and should not be altered for subsequent treatments. In the setup stage, the treatment location of a user must be accurately determined. This may be achieved using standard techniques as described above, such as described with respect to FIG. 1. Once this location is accurately determined, then the position of the coil 8 with respect to the first and second contact portions 12,14 is identified and recorded. It is important that the location of the coil 8 is accurately recorded in order that for each subsequent treatment the coil 8 is positioned at the same location.

Referring to FIG. 5, an alternative embodiment is presented where the second contact portion 14 is configured to contact a subject's ear. In this embodiment there are two second contact portions 14 both configured to contact the subject's ear and a first contact portion 12 for seating onto the bridge of a subject's nose. The three locations of both ears, and the bridge of the nose define a reference frame for positioning of the coil 8 (shown as a coil housing only in this schematic representation).

In this embodiment the first contact portion 12 seats onto the subject's nose bridge and extends via an intermediate portion 28 to each of the second contact portions 14 that seat around a subject's ear. A coil carrier 50 extends upwardly from the second contact portion 14 and forms a band over the head of a subject and is held in position by a corresponding second contact portion 14. This coil carrier 50 in the form of a band carries the coil via a sliding element 52 as best shown in FIG. 5b which can be locked in the treatment position as required meaning that the coil 8 is also locked into position. This allows adjustment of the position of the coil 8 across the subject's head. In addition, the coil 8 is adjustable forward and aft relative to the head of the subject by rotating at the interconnection between the band 50 and the second contact portions 14. As shown in FIG. 5c, indicia are provided for recording the alignment between the first and second contact portions 12,14 to ensure repeatable positioning.

An advantage of the apparatus of FIG. 5 is that it can be fixed in a predetermined configuration in a setup stage, and simply worn. With the fixed locations of both ears and nose bridge, the coil 8 is appropriately located in the treatment position repetitively with relative ease.

FIG. 6 is a further illustrative embodiment of the present invention similar to FIG. 5. In this embodiment again the second contact area is a subject's ear, but the second contact portion 14 is supported from below by the uppermost area where the ear projects from the head in the same area as for a pair of glasses. Again, in this embodiment the first contact portion 12 sits onto the bridge of a subject's nose. Band 50 is moveable fore and aft along the intermediate portion 28 extending between the first and second contact portions 12,14 thereby allowing appropriate positioning of the coil 8 fore and aft. Positioning of the coil 8 across the head is achieved in the same way as described with respect to FIG. 5, however the coil and sliding element 52 are not shown for clarity.

Monitoring equipment may afford a patient a near real-time feedback loop for coil alignment. In one embodiment, this may be with a series of video screens having indicators showing degree of alignment on up to six degrees of freedom (x, y, z, and three rotational angles). In another, it may have audible feedback with degree of alignment present in such a communication carrying the equivalent of analog information (with a pitch, for instance, that increased as alignment improves). In yet another embodiment, this feedback may be haptic or “touch” in nature, such as a gentle vibration being afforded when proper alignment is lost or confirmed.

A preferred environment is one in which a nearly-real time video image of the patient is available to the attending therapist, and a more preferred environment is one in which likewise a nearly-real time video image of the attending therapist is available to the patient. Both offer improved system safety and control, and, in the case of patients who may be unaware of or uncomfortable with an unknown medical process, a video image of a therapist may be particularly useful in minimizing patient discomfort.

The apparatus of any embodiment presented may be operated at different operating protocols and may treat the subject at different locations depending on the required effect and the protocol. Low frequency (eg around 1 Hz), high frequency (eg around 10 Hz) could be utilised, however for a TMA apparatus particularly suitable for home use treatment at low frequency is preferable as there is no requirement under this protocol for mechanical cooling of the coil such as with the use of fans.

Aspects of the present invention enable a new approach to delivery of TMS which is particularly but not exclusive suitable for delivery in a home environment.

In the first instance a psychiatrist may determine the medical appropriateness of TMS for a patient and discusses with the patient the option to receive TMS in the patient's home. The physician evaluates the willingness and ability of the patient to comply with the therapy requirements. The physician may then decide to prescribe TMS treatment.

The appropriate therapy protocol is then determined to ensure that the right treatment dose is determined for each patient and an apparatus is positioned relative to the patient's head that precisely aligns a treatment coil 8 with the intended treatment areas, the L-DLPFC (left dorsolateral prefrontal cortex). A medical professional then determines in a clinic environment the motor threshold of the patient with a TMS system utilizing the same protocol that is used today in any TMS centre. The patient might receive several sessions of TMS in this controlled environment prior to make sure they qualify for in-home use.

That patient takes the specialized coil and head appliance home. The patient also receives a patient computer device 18 that either contains specific TMS programming parameters or a link to a cloud based remote patient programming system.

A TMS therapy educator sets up the system at home and educates the patient on the use of the system. This apparatus includes the control system 18 that preferably controls the stimulator 2, tracks the patient's compliance and interfaces with the remote input device.

The subject may then undergo TMS guided by the control system 18 and the medical practitioner. The system measures compliance and queries the subject to self-report, such as reporting depression scores. The subject also has access to a call center that can answer technical questions and help a patient troubleshoot system issues and answer concerns relative to possible adverse events.

Post therapy follow up should be utilized where a physician assesses the patient's progress either via a tele-consult or during a patient visit. The physician can also monitor the patient's compliance and depression self-rating via the cloud-based patient control and management system. Ongoing therapy such as maintenance therapy may be prescribed by the physician and are entered remotely via the cloud-based patient control and management system.

Aspects and embodiments of the present invention have been described by way of example only and it will be appreciated to the skilled addressee that modifications and variations may be made without departing form the scope of protection afforded by the appended claims.

1. A Transcranial Magnetic Stimulation (TMS) system comprising a TMS apparatus for delivery of TMS to a subject and an input device physically disconnected from the TMS apparatus, the TMS apparatus comprising:
a coil comprising one or more coil windings wound from an elongate conductive element;
a stimulator for supplying electrical current to the one or more coil windings;
a control system for controlling the stimulator in delivery of a TMS treatment protocol to a subject, the control system comprising a communication unit configured to transmit information related to the subject and/or one or more operational parameters of the treatment protocol of the stimulator to the input device;
wherein the control system is further arranged to modify the treatment protocol in response to a control signal received from the input device.

2. A TMS system according to claim 1 wherein the control signal is dependent upon an input to the input device by a user remote from the TMS apparatus.

3. A TMS system according to any preceding claim wherein the stimulator supplies electrical current to the one or more coil windings in pulses, and wherein the operational parameters comprise one or more of pulse duration, pulse repetition rate, total number of pulses in a treatment protocol and maximum electrical current supplied to the one or more coil windings during each pulse.

4. A TMS system according to any preceding claim wherein the communication unit is arranged to transmit information related to the subject and/or one or more operational parameters of the treatment protocol of the stimulator to the input device during delivery of the treatment protocol.

5. A TMS system according to claim 4 wherein the control system is arranged to modify the treatment protocol in response to a control signal during delivery of the treatment protocol.

6. A TMS system according to any preceding claim wherein the control system is arranged to modify the treatment protocol by either terminating and/or modifying supply of electrical current to the one or more coil windings by the stimulator.

7. A TMS system according to any preceding claim arranged such that supply of electrical current from the stimulator to the one or more coil windings is only enabled when there is operative communication between the communication unit and the input device.

8. A TMS system according to any preceding claim arranged to maintain an operational connection between the communication unit and the input device, and only enable delivery of TMS to the subject when an operational connection is maintained.

9. A TMS system according to any preceding claim wherein the communication unit is arranged to transmit the information during delivery of a treatment protocol to a subject, the treatment protocol comprising delivery of one or more pulses of electrical current from the stimulator to the one or more coil windings, and one or more parameters of the treatment protocol is controlled dependent upon the information received by the device.

10. TMS system according to any preceding claim wherein the TMS apparatus comprises a subject operable switch moveable between an on and off position for enabling and disabling supplying of electrical current to the one or more coil windings, wherein the switch is arranged such that it must be actively maintained in an on position for the supplying of electrical current to the one or more coil windings.

11. A TMS system according to any preceding claim wherein the TMS apparatus comprises a camera for capturing visual images of the subject, and the information relating to the subject comprises captured image data.

12. A TMS system according to claim 11 wherein the captured images comprise moving visual images.

13. A TMS system according to any preceding claim wherein the input device comprises a display screen for displaying captured images of the subject.

14. A TMS system according to any preceding claim wherein the input device comprises a user operable input for receiving control instructions for the control system, and the control signal represents the control instructions.

15. A TMS system according to any preceding claim wherein the communication unit is arranged to encrypt the information before transmission to the input device.

16. A TMS System according to any preceding claim wherein the communication unit comprises a communication unit which is arranged to communicate with the input device over the internet.

17. A TMS system according to any preceding claim wherein the communication unit is arranged to communicate over a cellular network.

18. A TMS system according to any preceding claim wherein the TMS apparatus further comprises a subject operable input device for inputting information relating to the subject.

19. A TMS system according to any preceding claim wherein the TMS apparatus further comprises a monitoring arrangement for monitoring the subject during delivery of TMS to the subject and obtaining the information relating to the subject from the monitoring arrangement.

20. A TMS system according to claim 19 wherein the monitoring arrangement comprises an EEG and/or EMG monitoring arrangement and the information related to the subject comprises EEG data and/or EMG data.

21. A TMS system according to any preceding claim wherein the TMS apparatus further comprises a receiving formation for receiving a subject's head and for providing a reference frame for positioning the coil relative to a subject and a support structure for supporting the coil and receiving formation at an elevated position.

22. A TMS system according to any preceding claim wherein the receiving formation comprises a first contact portion for contacting a first area of a subject's head, a second contact portion for contacting a second area of a subject's head, and an intermediate portion extending between the first and second contact portions.

23. A TMS system according to any preceding claim wherein the support structure further comprises the stimulator.

24. A method of controlling delivery of Transcranial Magnetic Stimulation (TMS) to a subject comprising providing a TMS system according to any preceding claim and comprising the steps of:
transmitting information related to the subject and/or one or more operational parameters of the treatment protocol of the stimulator to the remote input device;
inputting TMS control instructions to the remote input device dependent on the transmitted information and transmitting a control signal to the communication unit of the TMS apparatus;
modifying the treatment protocol in response to the control signal received from the input device.

25. A method according to any claim 24 further comprising the step of a subject inputting information relating to the subject.

26. A method according to any of claims 24-25 further comprising the step of establishing an operational connection between the TMS apparatus and the input device, and only enabling delivery of the treatment protocol when the operative connection is maintained.

27. A method according to any of claims 24-25 wherein the information related to the subject and/or one or more operational parameters of the treatment protocol of the stimulator and the control signal are transmitted wirelessly over the internet.

28. A method according to any of claims 24-25 wherein the transmitted information comprises captured image data.

29. A Transcranial Magnetic Stimulation (TMS) system comprising a TMS apparatus for delivery of TMS to a subject and an input device physically disconnected from the TMS apparatus, the TMS apparatus comprising:
a coil comprising one or more coil windings wound from an elongate conductive element;
a stimulator for supplying electrical current to the one or more coil windings;
a control system for controlling the stimulator in delivery of a TMS treatment protocol to a subject;
wherein the system is arranged to establish and maintain an operational connection between the communication unit and the input device, and only enable delivery of TMS treatment to the subject when an operational connection is maintained.

30. A TMS system according to claim 29 wherein an operational connection is established in response to the control system receiving an authentication signal from the remote input device.

31. A TMS system according to any of claims 29-30 further comprising a camera for capturing moving visual image data of the subject and transmitting the moving visual image data to the TMS apparatus.

32. A TMS system according to according to any of claims 29-31 wherein the control system is arranged to modify the treatment protocol in response to a control signal received from the input device.

33. A TMS system according to any of claims 29-32 wherein the TMS apparatus comprises a subject operable switch moveable between an on and off position, wherein the switch is arranged such that it must be actively maintained in an on position for an operational connection to be maintained.

34. A method of controlling operation of a Transcranial Magnetic Stimulation (TMS), the method comprising providing a system according to any of claims 29-33, and establishing and maintaining an operational connection between the communication unit and the input device, and enabling delivery of TMS treatment to a subject only when an operational connection is maintained.

35. A Transcranial Magnetic Stimulation (TMS) apparatus comprising:
a coil comprising one or more coil windings wound from an elongate conductive element;
a receiving formation for receiving a subject's head and for providing a reference frame for positioning the coil relative to a subject; and
a support structure for supporting the coil and receiving formation at an elevated position.

36. A TMS apparatus according to claim 35 wherein the receiving formation comprises a first contact portion for contacting a first area of a subject's head, a second contact portion for contacting a second area of a subject's head, and an intermediate portion extending between the first and second contact portions.

37. A TMS apparatus according to any of claims 35-36 wherein the receiving formation comprises a third contact portion for contacting a third area of a subject's head.

38. A TMS apparatus according to any of claims 35-37 wherein the first and second areas are distinct areas of the subject's head.

39. A TMS apparatus according to claim 38 wherein the first area is a subject's chin or bridge of the nose and the second area is a subject's ear.

40. A TMS apparatus according to claim 36 wherein the second contact portion is shaped to at least partially seat into a subject's ear.

41. A TMS apparatus according to claim 36 wherein the second contact portion further comprises a speaker for outputting sound to the subject.

42. A TMS apparatus according to claim 36 wherein the first and/or second contact portions are demountable relative to the intermediate portion.

43. A TMS apparatus according to any of claims 35-42 wherein the support structure comprises an adjustment mechanism for adjusting and fixing the relative position between the coil and the receiving formation.

44. A TMS apparatus according to any of claims 33-41 wherein the support structure further comprises a display arrangement for displaying information relating to operation of the TMS apparatus.

45. A TMS apparatus according to any of claims 35-44 wherein the support structure further comprises a stimulator for supplying current to the one or more coil windings.

46. A TMS apparatus according to any of claims 35-45 wherein the support structure comprises a housing having a stimulator therein for supplying current to the one or more coil windings and a support arm arrangement extending therefrom for supporting the coil and the receiving formation.

47. A TMS apparatus according to claim 46 wherein the support arm arrangement extends intermediate the housing and the coil and receiving formation.

48. A TMS apparatus according to any of claims 46-47 wherein the support arm arrangement comprises multiple linkages enabling movement of the coil and receiving portion relative to the housing.

49. A TMS apparatus according to any of claims 46-48 wherein the support arm arrangement is configurable such that the receiving formation and coil are moveable between an operational configuration and a stowed configuration.

50. A TMS apparatus according to any of claims 46-49 wherein the support arrangement further comprises a display arrangement for displaying information relating to operation of the TMS apparatus.

51. A TMS apparatus according to any of claims receiving formation preferably defines a receiving zone open in a generally downward orientation when in an operative configuration.

52. Use of a TMS apparatus according to any of claims 37-53 wherein the support structure is supported on an elevated support surface.

53. Use of an apparatus according to claim 55 wherein a subject leans forwardly into the receiving formation and at least partially supports the weight of their upper body on the elevated support surface with their forearms.

54. A method of positioning a Transcranial Magnetic Stimulation (TMS) apparatus relative to a subject comprising:
providing a reference frame for positioning a coil relative to the subject by positioning a first area of a subject's head in contact with a first contact portion of a receiving formation and positioning a second contact area of a subject's head in contact with a second contact portion of the receiving formation;
positioning the coil at a predetermined treatment location in the reference frame.

55. A method according to claim 54 comprising finding the predetermined treatment location.

56. A positioning arrangement for enabling positioning of a TMS coil relative to a subject's head, the positioning arrangement comprising first and second contact portions for receiving a subject's head at a first and second contact area respectively and providing a reference frame for positioning a coil relative to the subject, the positioning arrangement further comprising a support structure for supporting the first and second contact portions, wherein at least the first contact portion is releasably mountable to the support structure.

57. A positioning arrangement according to claim 56 wherein both the first and second contact portions are releasably mountable to the support structure.

58. A positioning arrangement according to any of claims 56-57 wherein the first contact portion comprises a first contact pad and the second contact portion comprises a second contact pad.

59. A positioning arrangement according to any of claims 56-58 wherein first and preferably second contact portions are secured to the support arrangement by a securing arrangement.

60. A method of positioning a TMS coil relative to a subject's head using a positioning arrangement where the positioning arrangement comprises first and second contact portions for receiving a subject's head at a first and second contact area respectively and provide a reference frame for positioning a coil relative to the subject, wherein the method comprises recording the contours of at least the first contact area of the subject and using the recorded contours to form a customised first contact portion, and mounting the customised first contact portion to a support structure for supporting the first and second contact portions.
